(19) United States
(12) Patent Application Publication
(10) Pub. No.: US 2026/0102197 A1
(43) Pub. Date: Apr. 16, 2026

(54) CRYOABLATION DEVICES INCLUDING ECHOGENIC FEATURES, AND RELATED SYSTEMS AND METHODS

(71) Applicant: HOLOGIC, INC., Marlborough, MA (US)

(72) Inventors: Tarpit Patel, Farmington, CT (US); Ken DeFreitas, Patterson, NY (US)

(73) Assignee: HOLOGIC, INC., Marlborough, MA (US)

(21) Appl. No.: 19/117,245

(22) PCT Filed: Sep. 29, 2023
(86) PCT No.: PCT/US2023/075578

§ 371 Date: Mar. 31, 2025

Related U.S. Application Data
(60) Provisional application No. 63/378,011, filed on Sep. 30, 2022.

(51) Int. Cl. A61B 18/02; A61B 18/00; A61B 34/20

(52) U.S. Cl. CPC A61B 18/02; A61B 2018/00577; A61B 2018/0262; A61B 2034/2063

(57) ABSTRACT

A cryogenic probe includes a body having an active region configured to be inserted within a treatment region of a patient. The active region is defined by one or more internal passages configured to receive a flow of cryogenic fluid. The cryogenic probe body comprises one or more echogenic features on an exterior surface of the cryogenic probe body. The echogenic features are positioned to visually identify at least a portion of the active region under imaging. Disclosed devices, systems, and methods relate to cryoablation and imaging of treatment regions and cryogenic probes.

### CROSS-REFERENCE TO RELATED APPLICATIONS

This application claims priority to U.S. Provisional Application No. 63/378,011, filed Sep. 30, 2022, the entire contents of which is incorporated by reference herein.

### TECHNICAL FIELD

The present disclosure generally relates to the field of cryoablation devices and systems. More specifically, the disclosure relates to cryoablation devices and systems including echogenic features, such as cryoprobes with echogenic features or patterns.

### Introduction

Cryogen delivery systems, including systems with cryosurgical probes, are used to destroy diseased or abnormal tissue cells to treat a variety of medical conditions. When cryogenic liquids such as liquid nitrogen are used with a cryosurgical probe, tissues adjacent to the probe quickly freeze, causing the tissue to die, after which it is absorbed by the body, expelled by the body, or sloughed off. Cryogenic delivery systems can be used to treat, among other things, skin cancer, skin lesions, breast tumors (both benign and cancerous), prostate cancer and benign prostate disease, liver tumors and liver cancer, glaucoma and other eye diseases, and other conditions.

Cryogen delivery systems such as cryoablation devices can include a probe that receives a supply of a cryogen, such as liquid nitrogen or another cryogenic fluid. The probe may be inserted (e.g., subcutaneously) into a region containing a target tissue to be treated, such as, for example, a lesion or tumor. Cryogen flowing through the probe freezes the surrounding tissue, creating a generally spherical or oblong mass of frozen tissue (referred to as an “ice ball”). It is desired to freeze as much of the target tissue as possible while avoiding freezing healthy, unaffected tissue, so the location, orientation, and insertion depth of the probe are factors for the clinician carefully considers when placing the probe in the region to be treated. Further, once the cryogen begins flowing and the mass of frozen tissue begins to form, repositioning of the probe may be impractical or impossible because the probe is immobilized within the frozen mass. Additionally, ultrasound imagery of the probe and region to be treated may be impacted (e.g., partially or fully obscured) by formation of the frozen mass.

There is a need for devices and methods that facilitate insertion of the probe in the desired location for carrying out a cryoablation procedure that maximally affect the region to be treated while minimally impacting healthy tissue.

### SUMMARY

The present disclosure addresses one or more of the above-mentioned problems and/or achieves one or more of the above-mentioned desirable features. Other features and/or advantages may become apparent from the description which follows.

In accordance with various exemplary embodiments of the present disclosure, a cryogenic probe includes a body having a length and an active region. The active region is configured for insertion within a treatment region of a patient. The active region includes one or more internal passages configured to receive a flow of cryogenic fluid. The cryogenic probe also includes one or more echogenic features on an exterior surface of the body of the cryogenic probe. The one or more echogenic features are positioned to visually define at least a portion of the active region of the body of the cryogenic probe under imaging. In an example, the one or more echogenic features comprise a first echogenic feature proximate a midpoint of a length of the active region of the body of the cryogenic probe, a second echogenic feature proximate a proximal portion of the active region of the body of the cryogenic probe, and a third echogenic feature proximate a distal portion of the active region of the body of the cryogenic probe. In another example, the second echogenic feature and the third echogenic feature are equidistant from the first echogenic feature. In yet another example, the active region of the cryogenic probe comprises a distal, uninsulated portion of the body of the cryogenic probe. In a further example, a proximal end portion of the second echogenic feature is aligned with a proximal end of the active region of the body of the cryogenic probe and a distal end portion of the third echogenic feature is aligned with a distal end of the active region of the body of the cryogenic probe.

In another example, the first echogenic feature has a first width and the second and third echogenic features each have a second width different from the first width. In another example, the first width is less than the second width. In yet another example, the first echogenic feature has a first pattern and the second and third echogenic features each have a second pattern different from the first pattern. In a further example, one or more of a shape, density, or size of the first pattern differs from a shape, density, or size of the second pattern.

In another example, the one or more internal passages comprise a helical shape within at least a portion of the active region of the body of the cryogenic probe. In an additional example, the one or more echogenic features comprise a pattern etched into the exterior surface of the cryogenic probe. In a further example, the pattern comprises a repeating pattern of recessed dot elements.

In accordance with another aspect of the present disclosure, a cryosurgery system includes a cryogenic probe configured to be inserted within a treatment region of a patient. A body of the cryogenic probe has at least one echogenic feature on an exterior surface of the body. The at least one echogenic feature is configured to assist positioning of the probe under imaging. The system further comprises a cryogenic fluid supply and a cryogenic fluid supply line configured to provide a flow of cryogenic fluid to the cryogenic probe. In an example, the body of the cryogenic probe comprises an active region having one or more interior passages configured to receive a flow of cryogenic fluid. In another example, the at least one echogenic feature is located along at least a portion of the active region of the body of the cryogenic probe.

In another example, the system further comprises an imaging system with a display. In an example, the imaging system comprises an ultrasound imaging system.

In yet another example, the at least one echogenic feature comprises a repeating pattern of recessed dots. In example, the at least one echogenic feature comprises a first echogenic feature positioned at a midpoint of the active region of the body of the cryogenic probe and second and third echogenic features positioned at respective ends of the active region of the body of the cryogenic probe. In an additional example, the first echogenic feature has a first pattern and the second and third echogenic features each have a second pattern different from the first pattern. In a further example, one or more of a shape, density, or size of the first pattern differs from a shape, density, or size of the second pattern.

In accordance with another aspect of the present disclosure, a method of ablating a target tissue includes inserting a cryogenic probe into a treatment region of a patient. The cryogenic probe includes a body having an active region and one or more echogenic features on an exterior surface of the body of the cryogenic probe. The one or more echogenic features are positioned to visually define at least a portion of the active region of the body of the cryogenic probe. The method further includes imaging the treatment region and the cryogenic probe, displaying an image of the one or more echogenic features on the probe body and the treatment region on a display device, and positioning the cryogenic probe within the treatment region based on the display of the one or more echogenic features and the treatment region. In an example, imaging the treatment region further comprises imaging a target tissue present in the treatment region. In another example, the target tissue comprises a diseased tissue, an abnormal tissue, a fibroadenoma, a tumor, or a lesion.

In an example, positioning the cryogenic probe within the treatment region comprises positioning the cryogenic probe based on a location of the one or more echogenic features relative to the target tissue. In another example, positioning the cryogenic probe based on a location of the one or more echogenic features relative to the target tissue comprises moving the body of the cryogenic probe to position the target tissue adjacent to the one or more echogenic features. In yet another example, the one or more echogenic features comprise first and second echogenic features positioned at respective ends of an active region of the body of the cryogenic probe, and wherein moving the body of the cryogenic probe to position the target tissue adjacent to the one or more echogenic features includes centering the target tissue between the first and second echogenic features along the active region of the body of the cryogenic probe. In a further example, the method further includes circulating a cryogenic fluid through the active region of the body of the cryogenic probe to generate an ice ball around the target tissue and the active region of the body of the cryogenic probe.

In an additional example, imaging the treatment region comprises imaging the treatment region using ultrasound imaging. In a further example, the method further includes flowing a cryogenic fluid through passages defining an active region of the cryogenic probe. In yet another example, the method further includes freezing the target tissue within the treatment region via the cryogenic fluid flowing through the passages in the active portion of the cryogenic probe.

Additional objects and advantages will be set forth in part in the description which follows, and in part will be obvious from the description, or may be learned by practice of the present teachings. At least some of the objects and advantages of the present disclosure may be realized and attained by means of the elements and combinations particularly pointed out in the appended claims.

Both the foregoing general description and the following detailed description are exemplary and explanatory only and are not restrictive of the present disclosure and claims, including equivalents. The present disclosure and claims, in their broadest sense, could be practiced without having one or more features of these exemplary aspects and embodiments.

### BRIEF DESCRIPTION OF THE DRAWINGS

The accompanying drawings, which are incorporated in and constitute a part of this specification, illustrate some exemplary embodiments of the present disclosure and together with the description, serve to explain certain principles. These drawings depict only typical embodiments of the disclosed inventions and are not therefore to be considered limiting of its scope. In the drawings:

FIG. 1 is a perspective view of an example embodiment of a cryogen delivery system in accordance with the present disclosure.

FIG. 2 is a schematic view of a cryogen delivery system and an imaging system according to an exemplary embodiment of the present disclosure.

FIG. 3A is a schematic view of a cryogen probe inserted within a treatment region according to an exemplary embodiment of the present disclosure.

FIG. 3B is another schematic view of a cryogen probe within a treatment region and illustrating the temperature gradient formed around the active region of the cryoprobe.

FIG. 4 is a side view of a cryogenic probe according to an exemplary embodiment of the present disclosure.

FIG. 5 is a side view of the cryogenic probe of FIG. 4 inserted within a treatment region.

FIG. 6 is a partial, enlarged view of an echogenic feature of the cryogenic probe of FIG. 4.

FIG. 7 is a side view of another cryogenic probe according to an exemplary embodiment of the present disclosure.

FIG. 8 is a side view of an additional cryogenic probe according to another embodiment of the present disclosure.

FIG. 9 is a workflow showing a method of cryoablation according to an exemplary embodiment of the present disclosure.

FIGS. 10A and 10B are ultrasound images of a cryoprobe having echogenic features in accordance with the present disclosure.

### DETAILED DESCRIPTION OF THE ILLUSTRATED EMBODIMENTS

Exemplary embodiments of the present disclosure include cryogen delivery systems configured for medical treatment or surgical use, such as cryosurgical or cryoablation systems. Such systems may include, or be configured to be used in conjunction with, imaging systems, such as an ultrasound or other imaging system. The imaging system can provide the operator with a visual depiction of a treatment region in which the cryosurgical and/or cryoablation procedure will be carried out. According to exemplary embodiments herein, such cryogen delivery systems include features configured to provide information to an operator of the cryogen delivery system regarding the position of a cryogenic probe with respect to a portion of a target tissue in the treatment area. The target tissue may be, for example, a benign tissue such as a fibroadenoma or a diseased tissue, such as a cancerous lesion. The listing of benign and diseased tissues above are examples only and are not intended to limit the type of target tissue. The information thus provided to the operator can assist the operator in positioning the cryogenic probe relative to the portion of the target tissue, e.g., to ensure the entire portion of the target tissue is encompassed by an ice mass generated by the probe.

The cryogen delivery system can include a cryogenic probe, also referred to as a cryoprobe, comprising an active region. The active region of the probe can include one or more passages configured to receive a flow of cryogenic fluid and to freeze a region of tissue surrounding the active region of the probe. Due to various functional characteristics of imaging systems such as ultrasound imaging devices, the cryogenic probe may not be clearly defined or easily viewed in the image in all situations. Thus, the operator of the cryogen delivery system may be unable to accurately place the cryogenic probe in the treatment region or locate the probe through a central portion of the target tissue in a manner that results in complete ablation of the target tissue region. Ensuring that the target tissue is centered on the active region of the probe also reduces the chance that healthy tissue is inadvertently positioned within the active region of the cryoprobe.

According to exemplary embodiments herein, the cryogenic probe can include one or more features configured to be imaged by the imaging system and appear on a display accessible to an operator of the cryogen delivery system along with the visual depiction of the treatment region. Such features can improve the visibility of the cryogenic probe in the image provided by the imaging system, thereby facilitating the operator's ability to accurately place the cryogenic probe for improved (e.g., maximal) ablation of the target tissue areas.

In some exemplary embodiments, the one or more features can comprise echogenic features (i.e., features that are configured to reflect ultrasound waves) that correspond to the active region of the probe, that is the region of the probe capable of generating the frozen mass and achieving a temperature of −20° C. in order to kill the target tissue. The echogenic features can be configured to be clearly visible in an image provided by an ultrasonic imaging system, or other imaging systems. In some embodiments, the echogenic features can comprise visible markings on the cryoprobe such as an etched pattern in the surface of the probe, and the echogenic features can generally identify to the operator of the probe, when viewed via imaging, the active region of the probe. For example, according to one embodiment, the echogenic features can comprise, for example, a first echogenic feature located along a midpoint of the active region of the cryogenic probe around which the operator can center the target tissue. In a second example embodiment, the echogenic features can comprise, for example, a first echogenic feature located along a midpoint of the active region of the cryogenic probe, a second echogenic feature at a proximal portion of the active region of the cryogenic probe, and a third echogenic feature at a distal portion of the active region of the cryogenic probe. For example, in certain embodiments, the first and third echogenic features may be similar to or the same as one another to delineate the ends of the active region of the cryogenic probe while the second echogenic feature may be different from the first and third echogenic features in order to be easily distinguishable as different from those patterns indicating the ends of the active region of the cryogenic probe and to instead indicate a central portion of the active region of the cryogenic probe. In order to maximize the efficacy of the treatment of the target tissue, the operator of the system can use the echogenic features or markings to position the cryoprobe relative to the target tissue (e.g., target lesion) such that a center of the active region of the cryoprobe is located at a center of the target tissue. Ensuring that the target tissue is centered within the active region of the cryoprobe, which ensures that the target tissue will be encapsulated within the ice ball generated by the cryoprobe during treatment, results in freezing of the target tissue. In other embodiments, the echogenic features used to make the active region of the cryoprobe visible to the operator of the cryogenic system may comprise, for example, a gradient defined by varying size and/or density of the echogenic features or variation in other characteristics of the echogenic features, such as shape, depth, etc.

Exemplary embodiments of the present disclosure can be used with, for example, cryogen delivery systems commercialized by Hologic, Inc., Marlborough, MA, USA, and described in connection with U.S. Pat. No. 8,092,448 B2, (filed Apr. 27, 2007), and entitled “Cryosurgical System with Low Pressure Cryogenic Fluid Supply” and U.S. Pat. No. 9,050,072 B2 (filed Dec. 3, 2013) and entitled “Cryoprobe for Low Pressure Systems”, the entire contents of each of which are incorporated by reference herein.

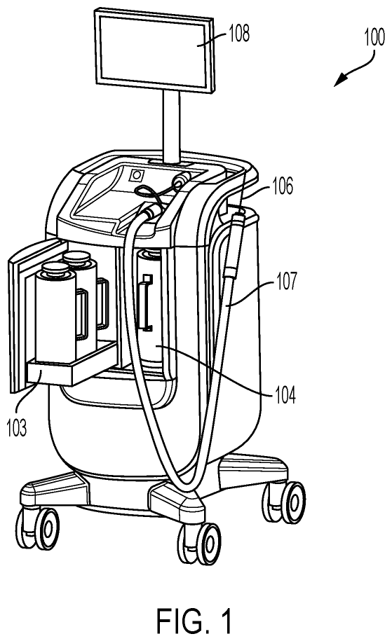

Referring now to FIG. 1, a cryogen delivery system 100 according to the present disclosure is shown. As shown in FIG. 1, the cryogen delivery system 100 can be in the form of a wheeled console to facilitate ease of use and portability. In some embodiments, the cryogen delivery system 100 can be configured to perform, for example, a cryosurgical procedure such as cryoablation. The cryogen delivery system comprises a probe 106, a cryogen supply hose 107 to which the probe 106 is coupled, and a cryogenic fluid supply vessel 104. The cryogen supply hose 107 is configured to receive a flow of cryogenic fluid from the cryogenic fluid supply vessel 104 for delivery through the probe 106 to a treatment location. The cryogen delivery system 100 includes a storage area 103 for storage of additional cryogenic fluid supply vessels. The cryogen delivery system 100 also includes a visual interface, such as a display 108, configured to communicate information to the operator of the cryogen delivery system 100 regarding the operational state of the cryogen delivery system 100. In some embodiments, the display 108 may comprise touchscreen controls to enable the operator to alter or input operational parameters and otherwise control the cryogen delivery system 100.

As discussed in more detail in U.S. Pat. No. 8,092,448, incorporated herein by reference above, a typical cryoablation procedure may include a series of alternating active freezing cycles, in which the cryogenic fluid is supplied to the cryoprobe 106 and target tissue is frozen using the probe 106, and passive thawing cycles in which the supply of cryogenic fluid to probe 106 is paused. One or more freezing cycles, alternated with passive thawing cycles, may be used to achieve the desired size of the ice ball used to treat the target tissue.

In some exemplary embodiments herein, the display 108 may be configured to display information related to the operational state of the cryogen delivery system, such as and not limited to, freeze/thaw cycle times, an amount of cryogenic fluid remaining in a Dewar or other cryogen reservoir, control elements configured to control start, end, and timing of a cryogenic procedure for which the cryogen delivery system is configured to be used. These examples are not limiting, and the display 108 can be used to provide other informational or control aspects related to operation of the cryogen delivery system.

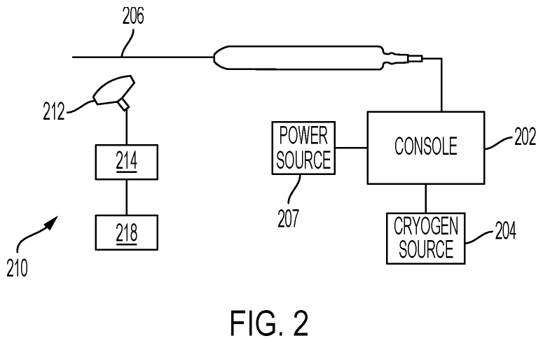

Referring now to FIG. 2, a schematic diagram of a cryogen delivery system and an imaging system 210 according to the present disclosure is shown. The cryogen delivery system includes a probe 206 operable coupled to a console 202 including or operably coupled to a cryogen source 204. The console 202 may be coupled to a power source 207 configured to provide electrical power to operate the cryogen delivery system. The imaging system includes an imaging sensor, such as an ultrasound transducer 212. The ultrasound transducer 212 is operably coupled to an imaging processing device, such as an ultrasound processor 214. The ultrasound processor 214 can include or be operably coupled to an imaging display 218. The imaging display 218 can provide a visual representation of the treatment region based on information collected by the ultrasound transducer 212 and processed by the ultrasound processor 214. It will be understood that the imaging system may be integral with the cryogen delivery system or separate from the cryogen deliver system. For example, in some integrated embodiments, the imaging system and cryogen delivery system may use the same display and/or processor or share other components. In separate embodiments, the imaging system may not share any components with the cryogen delivery system.

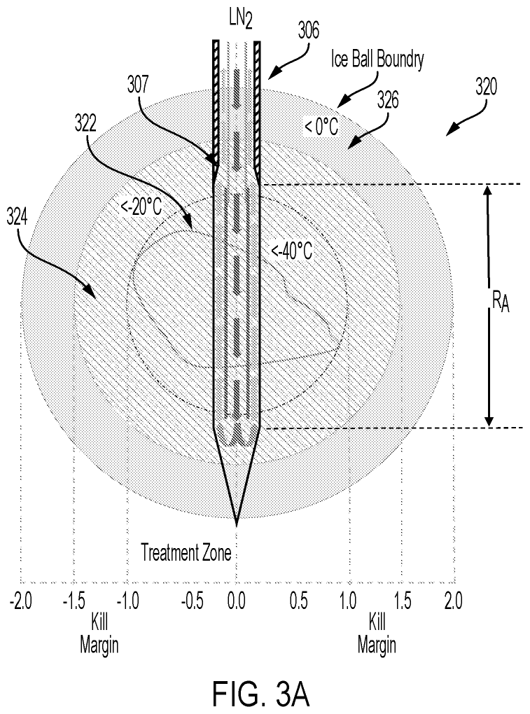

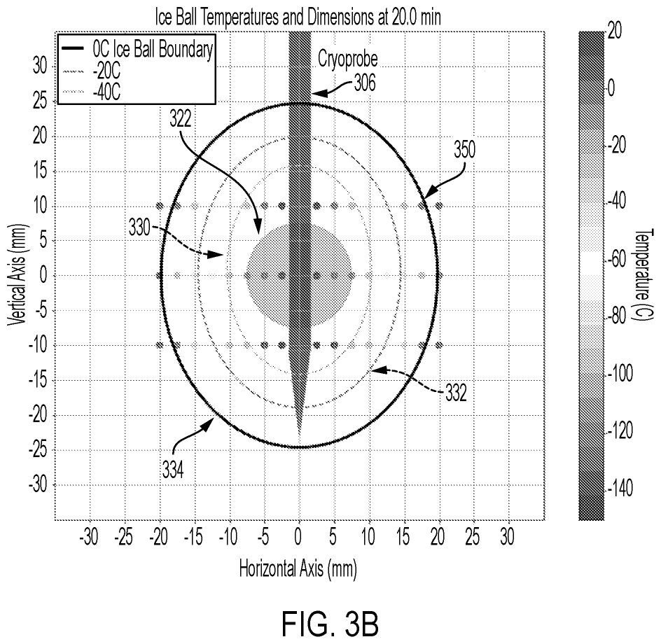

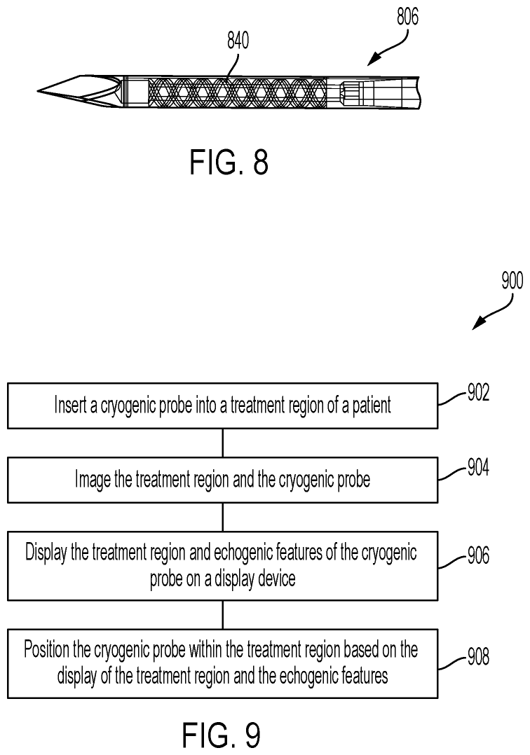

FIG. 3A is a schematic drawing showing a cryogenic probe 306 in an inserted position in a treatment region 320. The treatment region 320 includes a target tissue, in this exemplary embodiment the target tissue is a cancerous lesion 322. The cryogenic probe 306 includes an active region RA through which cryogen flows during use to cause freezing and ablation of the target tissue. As shown in FIG. 3A, the active region RA of the probe begins at a point where an insulated portion or insulation jacket 307 of the sheath ends. As shown in FIG. 3A, the freezing effects of the cryogenic probe 306 radiate outward from the active region RA of the cryogenic probe 306. In some embodiments, the freezing effects can be generally divided into zones that radiate outward from the cryogenic probe 306. For example, a “kill zone” 324 may be generated by operation of the cryogenic probe 306 and may extend a certain radius outward from the cryogenic probe 306. Tissue within the kill zone may be completely frozen such that cellular death is substantially certain for any tissue present within the kill zone 324. For example, the kill zone may encompass an area in which tissue reaches a temperature of between −20° C. and −40° C. (or colder). The size and shape of the kill zone 324 may vary based on the size and shape of the cryogenic probe and its internal baffle 840 (FIG. 8), the characteristics of the tissue within the treatment region, the length of time the cryogenic probe 306 is operating, and other factors. As noted above and discussed in more detail in U.S. Pat. No. 8,092,448, incorporated herein by reference above, one or more freezing cycles, alternated with passive thawing cycles, may be used to achieve the desired size of the ice ball used to treat the target tissue.

In the example embodiment of FIG. 3A, the kill zone 324 is depicted as generally circular and has a radius of approximately 1.5 cm outward from a midpoint of the active region R_{A} of the cryogenic probe 306. However, the kill zone 324 may vary in size and shape based on the various factors noted above, e.g., from about 1 cm in diameter to about 3 cm in diameter.

Outside the kill zone 324 lies a freeze zone 326. In the freeze zone, tissue may freeze to a certain extent and may be adversely affected, but cellular death may not be substantially certain. For example, the kill zone may encompass an area in which tissue reaches a temperature of between −0° C. and −20° C. The size and shape of the freeze zone depends on the same factors as discussed above in connection with the kill zone 324, and therefore, as the kill zone increases in size, the freeze zone 326 increases also. In the embodiment of FIG. 3A, the size of the freeze zone has a radius of approximately 2.0 cm outward from the center of the kill zone 324. In FIG. 3A, the freezing effects are shown generally circular for ease of illustration, but the actual shape of the various zones and freezing actions can be generally spherical, ellipsoidal, or may follow other general shapes based on the characteristics of the cryogenic probe 306 and the tissue characteristics of the treatment region 320. Further, although the kill zone 324 and freeze zone 326 are shown as being clearly delineated in FIG. 3A, a person of ordinary skill in the art will understand that actual temperatures and ice formation within the treatment region may form a gradient of increasing temperature away from the probe 306. Thus, the delineation between the kill zone 324 and freeze zone 326 may be a threshold temperature, such as the kill zone being defined as areas at and below a temperature of −20° C. based on dwell time and number of cycles, and the freeze zone being defines as areas at and below a temperature of 0° C. but above −20° C.

Because the tissue in the freeze zone 326 may be adversely affected (although not necessarily completely destroyed), it may be desired to create the smallest kill zone 324 that completely destroys the target tissue (e.g., cancerous lesion 322) to reduce (e.g., minimize) collateral damage to heathy tissue surrounding the target tissue. Further, because the kill zone and freeze zone extend generally radially outward from a midportion of the cryogenic probe 306, the location of the cryogenic probe 306 relative to the target tissue portion is important in minimizing damage to the surrounding tissue. That is, the more accurately the midportion of the cryogenic probe 306 can be located relative to a center of the target tissue, the smaller the size of a kill zone that will completely encompass the target tissue, and potentially, more healthy tissue will remain relatively unaffected.

FIG. 3B shows another example image of an ice ball 350 formed around the active region of probe 306. As shown in FIG. 3B, the target tissue comprises a cancerous lesion 322 is positioned in the center of ice ball 350. A generally oval line 330 identifies the outer edge of −40° C. temperature zone, which is the innermost ring of the kill zone 324. A second generally oval line 332 identifies the outer edge of the −20° C. temperature zone, which is the outer boundary of the kill zone 324. The generally oval line 334 identifies the outer boundary of the ice ball 350, and thus the 0° C. boundary. The area between lines 332 and 334 is generally referred to as the freeze zone 326.

As discussed above, the probe 306 may not always be clearly visible in the image of the treatment region provided by the imaging system (e.g., imaging system 210 shown in FIG. 2). Further complicating correct placement of the probe 306, once cryogenic fluid begins flowing through the probe 306, the probe 306 is quickly immobilized by formation of ice in the tissue being treated, so it is desired to accurately place the probe 306 at the beginning of the procedure. Accordingly, cryogenic probes of the present disclosure can include one or more features configured to increase visibility of the probe 306 in images provided by the imaging system 210 to aid in placement of the probe within the target tissue.

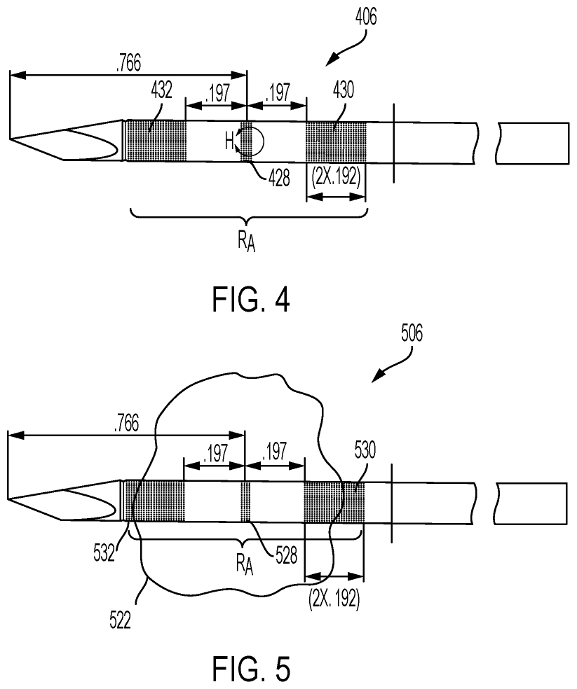

For example, referring now to FIG. 4, a cryogenic probe 406 is shown. The cryogenic probe 406 includes an active region RA along which cryogenic fluid acts to freeze adjacent tissue and form a kill zone and freeze zone as discussed above in connection with FIGS. 3A and 3B. The cryogenic probe comprises one or more features 428, 430, and 432 configured to increase visibility of the probe 406 within an image generated by an imaging system (e.g., 210 in FIG. 2). In the embodiment of FIG. 4, the one or more features 428, 430, and 432 comprise echogenic features, although other types of features may be used depending on the imaging technology used by the imaging system 210. In the exemplary embodiment of FIG. 4, the active region RA extends approximately 2 cm along the length of the probe 406. In other embodiments, the active region may be shorter or longer than 2 cm. The active region corresponds to a distal area of the cryogenic probe 406 that is uninsulated (see insulated portion or insulation jacket 307 of probe 306 and uninsulated active region RA of probe 306 in FIG. 3A) and contains an internal helical baffle 840 (see FIG. 8). The helical baffle is adapted to produce turbulent fluid flow in the fluid flowing through one or more passages in the cryoprobe. The baffle improves heat transfer by creating turbulent flow and forcing the cryogen to come in contact with the inner diameter surface of the outer wall in the active area of the cryoprobe.

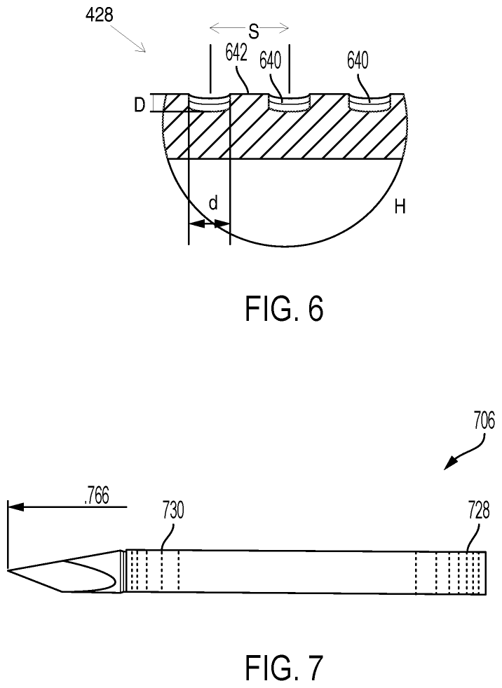

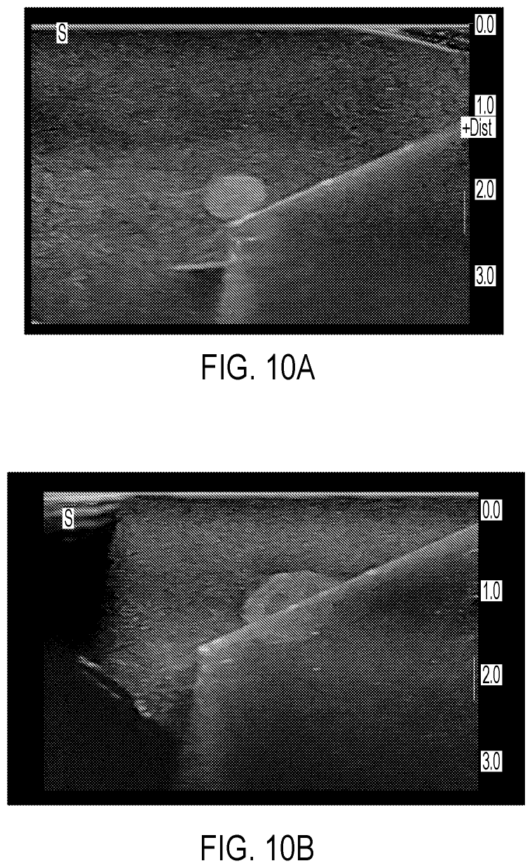

The one or more features 428, 430, and 432 may be configured to generally delineate the active region RA of the probe 406 within images generated by the imaging system 210. For example, the one or more features 428, 430, and 432 can include a first feature 428 positioned at a midpoint of the active region RA of the probe 406, a second feature 430 positioned at a proximal portion of the active region RA of the probe 406, and a third feature 432 positioned at a distal portion of the active region RA of the probe 406. In the exemplary embodiment of FIG. 4, the first feature 428, the second feature 430, and the third feature 432 comprise a repeating pattern of recessed dot elements extending around a circumference of the probe 406. In the exemplary embodiment of FIG. 4, a proximal end of the second feature 430 is aligned with a proximal end of the active region RA, and a distal end of the third feature 432 is aligned with a distal end of the active region RA. The specific shape, size, pattern, density, and other characteristics of each element of the one or more features 428, 430, and 432 can be chosen based on their ability to be imaged and subsequently displayed by the imaging system 210, as discussed further in connection with FIGS. 5 and 6. The one or more features may improve visibility of the probe 406 in an image based on the additional ultrasonic reflections, scattering, and other phenomena caused by the profiles of the one or more features on the probe 406. Parabolic reflector surfaces of the pattern creates a direct reflection of the transducer that enhances the visibility of the marking, in contrast to the smooth surface of needle cannula. Example ultrasonic images of a probe with features 428, 430, and 432 is shown in FIGS. 10A and 10B.

Various aspects and characteristics of the one or more echogenic features can be used to ensure the operator can clearly distinguish the midportion, proximal portion, and distal portion of the probe 406 from one another to further facilitate accurate placement of the probe 406 within the treatment region. For example, in the embodiment of FIG. 4, the second feature 430 and third feature 432 each extend along a larger portion of the probe 406 than does the first feature 428. Thus, the operator can recognize, e.g., in an image provided by the imaging system 210, the relatively narrow band of the first feature 428 as compared to the wider bands of the second feature 430 and third feature 432 to accurately place the probe 406 within the treatment region. Other variations in characteristics of the one or more features can be used to distinguish between the first, second and third features. For example, the size, shape, density, and/or pattern of individual elements of the features can vary between the first, second and third features to facilitate distinguishing between the midpoint, proximal portion, and distal portion of the active region RA of the probe 406.

Referring now to FIG. 5, a cryogenic probe 506 similar to probe 406 (FIG. 4) is shown inserted into a treatment region 534 comprising a target tissue region is shown. In FIG. 5, the target tissue region comprises a cancerous lesion 522. As shown in FIG. 5, a first feature 528 of the cryogenic probe 506 can be generally placed at a central location (e.g., centroid) of the cancerous lesion 522. Because the target tissue to be treated by the cryogenic probe 506 may be irregular in shape, a second feature 530 corresponding to a proximal portion of an active region RA of the probe 506 and a third feature corresponding to a distal portion of the active region RA of the probe 506 can also be used to position the cryogenic probe 506 appropriately so the kill zone (e.g., kill zone 324 shown in FIG. 3A) encompasses the entire cancerous lesion 522 without the freeze zone (e.g., freeze zone 326 shown in FIG. 3A) becoming unduly large and affecting a significant portion of healthy tissue. In this way, the first feature 528, second feature 530, and third feature 532 can provide the operator with information to accurately place the probe 506 within the treatment region.

Referring now to FIG. 6, a partial, enlarged view of the first echogenic feature 428 in FIG. 4 is shown. In the embodiment of FIG. 6, each individual element of the overall echogenic feature 428 comprises a recessed dot element 640. Although not shown for simplicity, each individual element of each of the overall second echogenic feature 430 and third echogenic feature 432 comprise similar dot elements 640. In this exemplary embodiment, each of the recessed dot elements 640 has a diameter d of approximately 0.1 mm, a depth D of approximately 0.04 mm, and a center-to-center spacing S of 0.2 mm. Variations to the shape, size, depth, spacing, and pattern of each of the elements 640 are within the scope of the present disclosure. For example, while the elements 640 may be generally round in the exemplary embodiment of FIG. 6, in other embodiments, the elements 640 may be ellipsoid, square, rectangular, rhomboid, etc. Further, variations in spacing, density, size, and shape can be used to distinguish various portions of the active region of the probe. The elements 640 can be formed in a surface 642 of the probe by any suitable method. For example, the probe may comprise a metal or metal alloy, such as stainless steel, and the elements 640 may be formed by laser etching, chemical etching, mechanical etching (e.g., media blasting) or other methods.

In the embodiments shown in FIGS. 4 and 5, the cryogenic probe 406, 506 includes a first feature 428, 528, a second feature 430, 530, and a third feature 432, 532. As discussed above in connection with those drawings, the three features together can provide the cryogen delivery system information, such as images, related to the positioning of the probe within the treatment region to enhance the operator's ability to accurately target tissue for treatment. Other configurations of features are within the scope of the disclosure. For example, in some embodiments, the features may include only features at the proximal and distal ends of the active region of the probe. In some embodiments, the features may form a gradient using, for example, varying size, shape, and/or density of elements of the features, and the gradient may be configured to indicate the active region of the probe in images generated by the imaging system. For example, with reference to FIG. 7, a probe 706 is shown with a first feature 728 at a proximal end portion of an active region RA and a second feature 730 at a distal end portion of the active region RA. The first feature 728 comprises a gradient with decreasing density of elements in a distal direction, and the second feature 730 comprises a gradient with decreasing density of elements in a proximal direction. The first feature 728 and second feature 730 thereby delineate for the operator the proximal and distal ends of the active region RA and facilitate placement of the probe 706 within a treatment region as discussed above. The embodiment of FIG. 7 does not include a feature at a midpoint of the active region RA, but such a feature could optionally be included.

The present disclosure also contemplates that additional markings may be used on various parts of the cryoprobe to communicate other information to the operator, such as, for example, the depth of the needle within the patient. To avoid confusion, different types of markings may be used to convey different types of information to the operator. Additionally, or alternatively, it is possible that a lack of markings in a certain area of the probe may also convey information to the operator regarding positioning or depth of the probe.

As noted herein, the active region of the cryogenic probe may include passages through which a cryogen flows and draws heat from surrounding tissue to freeze the target tissue desired to be ablated. In order to increase thermal exposure of the tissue to the cryogen, the active region of the cryogenic probe may include passages formed in a helical shape. For example, referring to FIG. 8, a perspective view of a cryogenic probe 806 is shown. Passages 840 within an active region of the cryogenic probe 806 are helically shaped to increase heat transfer from the tissue surrounding the probe to the cryogen flowing within the probe 806.

Cryogenic probes according to exemplary embodiments herein can include helical passages in accordance with the disclosure of U.S. Pat. No. 9,050,072, incorporated by reference above. However, the present disclosure is not limited to helical passages, and other shapes and configurations of active regions of probes are within the scope of the present disclosure.

Referring now to FIG. 9, a workflow 900 for carrying out a cryogenic procedure, such as cryoablation, is shown. At 902, a cryogenic probe is inserted into a treatment region of a patient. The cryogenic probe comprises an active region and one or more echogenic features along at least a portion of the active region. At 904, the treatment region and the cryogenic probe are imaged, e.g., by an imaging system. At 906, the treatment region and echogenic features of the cryogenic probe are displayed on a display device. At 908, the cryogenic probe is positioned within the treatment region based on the display of the treatment region and the echogenic features.

In one example embodiment of performing a cryogenic procedure to treat a fibroadenoma using the workflow of FIG. 9, the target tissue is a benign breast tumor or fibroadenoma that extends for about 2 cm along the longest length of the tumor. The cryogenic system uses a cryogenic probe in accordance with the probes disclosed and discussed above (probe 106, 206, 306, 406, 506). The cryogenic probe comprises an active region that corresponds to a distal area of the cryogenic probe that is uninsulated and includes one or more echogenic features along at least a portion of the active region. Using an imaging system (which may be integral with or separate from the cryogenic system), the clinician or system operator positions the probe such that the longest length of the fibroadenoma (2 cm) is positioned along the active region of the probe and positions the fibroadenoma relative to the one or more echogenic features on the active region of the probe.

For example, in one embodiment, using ultrasound imaging for guidance, the clinician or system operator positions the probe such that the longest length of the fibroadenoma is centered along the probe with respect to an echogenic feature that denotes a central region or a midpoint of the active region of the probe. In another example embodiment, using ultrasound imaging for guidance, the clinician or system operator positions the probe such that the longest length of the fibroadenoma is centered along the probe between first and second echogenic features that denote first and second end regions, respectively, of the active region of the probe. In yet another example embodiment, using ultrasound imaging for guidance, the clinician or system operator positions the probe such that the longest length of the fibroadenoma is centered along the probe between first and second echogenic features that denote first and second end regions, respectively, of the active region of the probe and also positions the probe relative to the tumor such that the tumor is centered around a third echogenic feature that denotes a central region or midpoint of the active region of the probe.

After positioning of the fibroadenoma relative to the active region of the probe is confirmed with imaging, one or more freeze cycles, alternating with passive thawing cycles, are run. The size of the ice ball generated during the freeze cycles can be monitored via the ultrasound imaging, with the clinician or system operator able to view of the size of the ice ball relative to the echogenic marker(s) on the probe. The cycles are repeated until the fibroadenoma is a frozen mass within an ice ball generated by the probe, with the kill zone generated by the probe extending to or beyond the borders of the fibroadenoma. Additional detail regarding the specific process of the use of alternating freeze and thaw cycles, such as length of cycles, size of ice ball, and completion of treatment may be found in U.S. Pat. No. 8,092,448, which is incorporated herein by reference.

In one example embodiment of performing a cryogenic procedure to treat breast cancer using the workflow of FIG. 9, the target tissue is a breast cancer tumor that extends for about 2 cm along the longest length of the tumor. The cryogenic system uses a cryogenic probe in accordance with the probes disclosed and discussed above (probe 106, 206, 306, 406, 506). The cryogenic probe comprises an active region that corresponds to a distal area of the cryogenic probe that is uninsulated and includes one or more echogenic features along at least a portion of the active region. Using an imaging system (which may be integral with or separate from the cryogenic system), the clinician or system operator positions the probe such that the longest length of the breast cancer tumor (2 cm) is positioned along the active region of the probe and positions the target tissue (cancerous tumor) relative to the one or more echogenic features on the active region of the probe.

For example, in one embodiment, using ultrasound imaging for guidance, the clinician or system operator positions the probe such that the longest length of the tumor is centered along the probe with respect to an echogenic feature that denotes a central region or a midpoint of the active region of the probe. In another example embodiment, using ultrasound imaging for guidance, the clinician or system operator positions the probe such that the longest length of the breast cancer tumor is centered along the probe between first and second echogenic features that denote first and second end regions, respectively, of the active region of the probe. In yet another example embodiment, using ultrasound imaging for guidance, the clinician or system operator positions the probe such that the longest length of the breast cancer tumor is centered along the probe between first and second echogenic features that denote first and second end regions, respectively, of the active region of the probe and also positions the probe relative to the breast cancer tumor such that the tumor is centered around a third echogenic feature that denotes a central region or midpoint of the active region of the probe.

After positioning of the fibroadenoma relative to the active region of the probe is confirmed with imaging, one or more freeze cycles, alternating with passive thawing cycles, are run. The size of the ice ball generated during the freeze cycles can be monitored via the ultrasound imaging, with the clinician or system operator able to view of the size of the ice ball relative to the echogenic marker(s) on the probe. The cycles are repeated until the breast cancer tumor is a frozen mass within an ice ball generated by the probe, with the kill zone generated by the probe extending to or beyond the borders of the breast cancer tumor.

Additional detail regarding the specific process of the use of alternating freeze and thaw cycles, such as length of cycles, size of ice ball, and completion of treatment may be found in U.S. Pat. No. 8,092,448, which is incorporated herein by reference.

Devices and systems according to the present disclosure can be used, for example, for cryoablation procedures used for treatment of both benign and cancerous tumors. Systems according to the present disclosure can increase the efficiency and efficacy of cryoablation procedures by providing useful information to the operator of the cryogen delivery system regarding the position of the cryogenic probe with respect to a treatment region.

### EXAMPLES

Illustrative examples of the cryogenic probes, cryosurgery systems, and methods of ablating a target tissue are provided below. Embodiments of the cryogenic probes, cryosurgery systems and methods described herein may include any one or more, and any combination of, the clauses described below:

Clause 1. A cryogenic probe, comprising:
a body having a length;
an active region of the body of the cryogenic probe, the active region being configured for insertion within a treatment region of a patient, the active region comprising one or more internal passages configured to receive a flow of cryogenic fluid; and
one or more echogenic features on an exterior surface of the body of the cryogenic probe, the one or more echogenic features being positioned to visually define at least a portion of the active region of the body of the cryogenic probe under imaging.

Clause 2. The cryogenic probe of clause 1, wherein the one or more echogenic features comprise a first echogenic feature proximate a midpoint of a length of the active region of the body of the cryogenic probe, a second echogenic feature proximate a proximal portion of the active region of the body of the cryogenic probe, and a third echogenic feature proximate a distal portion of the active region of the body of the cryogenic probe.

Clause 3. The cryogenic probe of clause 2, wherein the second echogenic feature and the third echogenic feature are equidistant from the first echogenic feature.

Clause 4. The cryogenic probe of clause 3, wherein the active region of the cryogenic probe comprises a distal, uninsulated portion of the body of the cryogenic probe.

Clause 5. The cryogenic probe of any one of clauses 2-4, wherein a proximal end portion of the second echogenic feature is aligned with a proximal end of the active region of the body of the cryogenic probe and a distal end portion of the third echogenic feature is aligned with a distal end of the active region of the body of the cryogenic probe.

Clause 6. The cryogenic probe of any one of clauses 2-5, wherein the first echogenic feature has a first width and the second and third echogenic features each have a second width different from the first width.

Clause 7. The cryogenic probe of clause 6, wherein the first width is less than the second width.

Clause 8. The cryogenic probe of any one of clauses 2-7, wherein the first echogenic feature has a first pattern and the second and third echogenic features each have a second pattern different from the first pattern.

Clause 9. The cryogenic probe of clause 8, wherein one or more of a shape, density, or size of the first pattern differs from a shape, density, or size of the second pattern.

Clause 10. The cryogenic probe of any one of clauses 1-9, wherein the one or more internal passages comprise a helical shape within at least a portion of the active region of the body of the cryogenic probe.

Clause 11. The cryogenic probe of any one of clauses 1-10, wherein the one or more echogenic features comprise a pattern etched into the exterior surface of the cryogenic probe.

Clause 12. The cryogenic probe of any one of clauses 8-11, wherein the pattern comprises a repeating pattern of recessed dot elements.

Clause 13. A cryosurgery system, comprising:
a cryogenic probe configured to be inserted within a treatment region of a patient, a body of the cryogenic probe having at least one echogenic feature on an exterior surface of the body of the cryogenic probe, the at least one echogenic feature being configured to assist positioning of the cryogenic probe under imaging;
a cryogenic fluid supply; and
a cryogenic fluid supply line configured to provide a flow of cryogenic fluid to the cryogenic probe.

Clause 14. The system of clause 13, wherein the body of the cryogenic probe comprises an active region having one or more interior passages configured to receive a flow of cryogenic fluid.

Clause 15. The system of clause 13 or clause 14, wherein the at least one echogenic feature is located along at least a portion of the active region of the body of the cryogenic probe.

Clause 16. The system of any one of clauses 13-15, further comprising an imaging system with a display.

Clause 17. The system of clause 16, wherein the imaging system comprises an ultrasound imaging system.

Clause 18. The system of any of clauses 13-17, wherein the at least one echogenic feature comprises a repeating pattern of recessed dots.

Clause 19. The system of any one of clauses 14-18, wherein the at least one echogenic feature comprises a first echogenic feature positioned at a midpoint of the active region of the body of the cryogenic probe and second and third echogenic features positioned at respective ends of the active region of the body of the cryogenic probe.

Clause 20. The system of clause 19, wherein the first echogenic feature has a first pattern and the second and third echogenic features each have a second pattern different from the first pattern.

Clause 21. The system of clause 20, wherein one or more of a shape, density, or size of the first pattern differs from a shape, density, or size of the second pattern.

Clause 22. A method of ablating a target tissue, comprising:
inserting a cryogenic probe into a treatment region of a patient, the cryogenic probe comprising a body having an active region and one or more echogenic features on an exterior surface of the body of the cryogenic probe, the one or more echogenic features being positioned to visually define at least a portion of the active region of the body of the cryogenic probe;
imaging the treatment region and the cryogenic probe;
displaying an image of the one or more echogenic features on the body of the cryogenic probe and the treatment region on a display device; and
positioning the cryogenic probe within the treatment region based on the display of the one or more echogenic features and the treatment region.

Clause 23. The method of clause 22, wherein imaging the treatment region further comprises imaging a target tissue present in the treatment region.

Clause 24. The method of clause 23, wherein the target tissue comprises a diseased tissue, an abnormal tissue, a fibroadenoma, a tumor, or a lesion.

Clause 25. The method of any one of clauses 22-24, wherein positioning the cryogenic probe within the treatment region comprises positioning the cryogenic probe based on a location of the one or more echogenic features relative to the target tissue.

Clause 26. The method of clause 25, wherein positioning the cryogenic probe based on a location of the one or more echogenic features relative to the target tissue comprises moving the body of the cryogenic probe to position the target tissue adjacent to the one or more echogenic features.

Clause 27. The method of clause 26, wherein the one or more echogenic features comprise first and second echogenic features positioned at respective ends of an active region of the body of the cryogenic probe, and wherein moving the body of the cryogenic probe to position the target tissue adjacent to the one or more echogenic features includes centering the target tissue between the first and second echogenic features along the active region of the body of the cryogenic probe.

Clause 28. The method of any one of clauses 23-27, further comprising circulating a cryogenic fluid through the active region of the body of the cryogenic probe to generate an ice ball around the target tissue and the active region of the body of the cryogenic probe.

Clause 29. The method of any one of clauses 22-28, wherein the imaging the treatment region comprises imaging the treatment region using ultrasound imaging.

Clause 30. The method of any one of clauses 22-29, further comprising flowing a cryogenic fluid through passages defining an active region of the cryogenic probe.

Clause 31. The method of clause 30, further comprising freezing the target tissue within the treatment region via the cryogenic fluid flowing through the passages in the active portion of the cryogenic probe.

This disclosure described some examples of the present technology with reference to the accompanying drawings, in which some of the possible examples were shown. Other aspects can, however, be embodied in many different forms and should not be construed as limited to the examples set forth herein. Rather, these examples were provided so that this disclosure was thorough and complete and fully conveyed the scope of the possible examples to those skill in the art.

The cryogenic probe of any one of clauses 1-12 can be used with the cryosurgery system of any one of clauses 13-21 and/or the cryogenic probe of any one of clauses 1-12 can be used to practice the method recited in any one of the clauses 22-31.

The cryosurgery system of any one of clauses 13-21 can incorporate the cryogenic probe of any one of clauses 1-12 and/or the cryosurgery system of any one of clauses 13-21 can be used to practice the method recited in any one of clauses 22-31.

The method of clauses 22-31 can be practiced with the cryogenic probe of any one of clauses 1-12 and/or the cryosurgery system of any one of claims 13-21.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

This description and the accompanying drawings that illustrate exemplary embodiments should not be taken as limiting. Various mechanical, compositional, structural, electrical, and operational changes may be made without departing from the scope of this description and the claims, including equivalents. In some instances, well-known structures and techniques have not been shown or described in detail so as not to obscure the disclosure. Furthermore, elements and their associated features that are described in detail with reference to one embodiment may, whenever practical, be included in other embodiments in which they are not specifically shown or described. For example, if an element is described in detail with reference to one embodiment and is not described with reference to a second embodiment, the element may nevertheless be included in the second embodiment.

It is noted that, as used herein, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to limit the disclosure. For example, spatially relative terms—such as “beneath,” “below,” “lower,” “above,” “upper,” “forward,” “front,” “behind,” and the like—may be used to describe one element's or feature's relationship to another element or feature as illustrated in the orientation of the figures. These spatially relative terms are intended to encompass different positions and orientations of a device in use or operation in addition to the position and orientation shown in the figures. For example, if a device in the figures is inverted, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly.

Further modifications and alternative embodiments will be apparent to those of ordinary skill in the art in view of the disclosure herein. For example, the systems may include additional components that were omitted from the diagrams and description for clarity of operation. Accordingly, this description is to be construed as illustrative only and is for the purpose of teaching those skilled in the art the general manner of carrying out the systems and methods of the present disclosure. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the scope of the present disclosure.

It is to be understood that the particular examples and embodiments set forth herein are non-limiting, and modifications to structure, dimensions, materials, and methodologies may be made without departing from the scope of the present disclosure. Other embodiments in accordance with the present disclosure will be apparent to those skilled in the art from consideration of the specification and practice of the invention disclosed herein. It is intended that the specification and examples be considered as exemplary only, with being entitled to their full breadth of scope, including equivalents.

1. A cryogenic probe, comprising:
a body having a length;
an active region of the body, the active region being configured for insertion within a treatment region of a patient, the active region comprising one or more internal passages configured to receive a flow of cryogenic fluid; and
one or more echogenic features on an exterior surface of the body of the cryogenic probe, the one or more echogenic features being positioned to visually define at least a portion of the active region of the body of the cryogenic probe under imaging.

2. The cryogenic probe of claim 1, wherein the one or more echogenic features comprise a first echogenic feature proximate a midpoint of a length of the active region of the body of the cryogenic probe, a second echogenic feature proximate a proximal portion of the active region of the body of the cryogenic probe, and a third echogenic feature proximate a distal portion of the active region of the body of the cryogenic probe.

3. The cryogenic probe of claim 2, wherein the second echogenic feature and the third echogenic feature are equidistant from the first echogenic feature.

4. The cryogenic probe of claim 3, wherein the active region of the cryogenic probe comprises a distal, uninsulated portion of the body of the cryogenic probe.

5. The cryogenic probe of claim 2, wherein a proximal end portion of the second echogenic feature is aligned with a proximal end of the active region of the body of the cryogenic probe and a distal end portion of the third echogenic feature is aligned with a distal end of the active region of the body of the cryogenic probe.

6. The cryogenic probe of claim 2, wherein the first echogenic feature has a first width and the second and third echogenic features each have a second width different from the first width, the first width being less than the second width.

7. (canceled)

8. The cryogenic probe of claim 2, wherein the first echogenic feature has a first pattern and the second and third echogenic features each have a second pattern different from the first pattern.

9. The cryogenic probe of claim 8, wherein one or more of a shape, density, or size of the first pattern differs from a shape, density, or size of the second pattern.

10. The cryogenic probe of claim 1, wherein the one or more internal passages comprise a helical shape within at least a portion of the active region of the body of the cryogenic probe.

11. The cryogenic probe of claim 1, wherein the one or more echogenic features comprise a pattern etched into the exterior surface of the cryogenic probe.

12. The cryogenic probe of claim 8, wherein the pattern comprises a repeating pattern of recessed dot elements.

13. A cryosurgery system, comprising:
a cryogenic probe configured to be inserted within a treatment region of a patient, a body of the cryogenic probe having at least one echogenic feature on an exterior surface of the body, the at least one echogenic feature being configured to assist positioning of the cryogenic probe under imaging;
a cryogenic fluid supply; and
a cryogenic fluid supply line configured to provide a flow of cryogenic fluid to the cryogenic probe.

14. The system of claim 13, wherein the body of the cryogenic probe comprises an active region having one or more interior passages configured to receive a flow of cryogenic fluid, and wherein the at least one echogenic feature is located along at least a portion of the active region of the body of the cryogenic probe. Claims 15.-18. (canceled)

19. The system of claim 13, wherein the at least one echogenic feature comprises a first echogenic feature positioned at a midpoint of the active region of the body of the cryogenic probe and second and third echogenic features positioned at respective ends of the active region of the body of the cryogenic probe.

20. The system of claim 19, wherein the first echogenic feature has a first pattern and the second and third echogenic features each have a second pattern different from the first pattern.

21. (canceled)

22. A method of ablating a target tissue, comprising:
inserting a cryogenic probe into a treatment region of a patient, the cryogenic probe comprising a body having an active region and one or more echogenic features on an exterior surface of the body of the cryogenic probe, the one or more echogenic features being positioned to visually define at least a portion of the active region of the body of the cryogenic probe;
imaging the treatment region and the cryogenic probe;
displaying an image of the one or more echogenic features on the body of the cryogenic probe and the treatment region on a display device; and
positioning the cryogenic probe within the treatment region based on the display of the one or more echogenic features and the treatment region.

23. The method of claim 22, wherein imaging the treatment region comprises imaging a target tissue present in the treatment region.

24. (canceled)

25. The method of claim 22, wherein positioning the cryogenic probe within the treatment region comprises positioning the cryogenic probe based on a location of the one or more echogenic features relative to the target tissue.

26. (canceled)

27. The method of claim 25, wherein the one or more echogenic features comprise first and second echogenic features positioned at respective ends of an active region of the body of the cryogenic probe, and wherein moving the body of the cryogenic probe to position the target tissue adjacent to the one or more echogenic features includes centering the target tissue between the first and second echogenic features along the active region of the body of the cryogenic probe.

28. (canceled)

29. (canceled)

30. The method of claim 22 any one of claims 22-29, further comprising
flowing a cryogenic fluid through passages defining an active region of the cryogenic probe, and
freezing a target tissue within the treatment region via the cryogenic fluid flowing through the passages in the active portion of the cryogenic probe.

31. (canceled)
